(19) United States
(12) Patent Application Publication
(10) Pub. No.: US 2013/0211537 A1
(43) Pub. Date: Aug. 15, 2013

(54) MEDICAL DEVICES INCLUDING DUPLEX STAINLESS STEEL

(71) Applicants: Gurpreet S. Sandhu, Rochester, MN (US); Robert D. Simari, Rochester, MN (US); David R. Holmes, JR., Rochester, MN (US); Dan Dragomir-Daescu, Rochester, MN (US); Anthony P. Parakka, Rockford, IL (US)

(72) Inventors: Gurpreet S. Sandhu, Rochester, MN (US); Robert D. Simari, Rochester, MN (US); David R. Holmes, JR., Rochester, MN (US); Dan Dragomir-Daescu, Rochester, MN (US); Anthony P. Parakka, Rockford, IL (US)

(73) Assignee: MAYO FOUNDATION FOR MEDICAL EDUCATION AND RESEARCH, Rochester, MN (US)

(21) Appl. No.: 13/699,477

(22) PCT Filed: May 24, 2011
(86) PCT No.: PCT/US11/37707

§ 371 Date: Feb. 1, 2013

Related U.S. Application Data
(60) Provisional application No. 61/347,665, filed on May 24, 2010.

(51) Int. Cl. A61F 2/82

(52) U.S. Cl. CPC A61F 2/82
USPC 623/23.7

(57) ABSTRACT

Medical devices, such as endoprostheses, and methods of making the devices are disclosed. The endoprostheses comprise a tubular member capable of maintaining patency of a bodily vessel. The tubular member includes a duplex stainless steel (e.g., 2205 stainless steel), which may be magnetized.

### RELATED APPLICATION

The present application claims the benefit under 35 U.S.C. §119(e) of U.S. Provisional Patent Application No. 61/347,665, filed on May 24, 2010 and titled MEDICAL DEVICES INCLUDING DUPLEX STAINLESS STEEL, which is hereby incorporated by reference in its entirety.

Medical devices, such as endoprostheses (e.g., stents) constructed of duplex stainless steels are described herein.

Human and other animal bodies includes various passageways such as arteries, other blood vessels, and other body lumens. These passageways sometimes become occluded or weakened. For example, the passageways may be occluded by a tumor, restricted by plaque, or weakened by an aneurysm. When this occurs, the passageway may be reopened or reinforced, or even replaced, with a medical endoprosthesis. An endoprosthesis is typically a tubular member that is placed in a lumen in the body. Examples of endoprostheses include stents, covered stents, and stent-grafts.

Endoprostheses may be delivered inside the body by a catheter that supports the endoprosthesis in a compacted or reduced-size form as the endoprosthesis is transported to a desired site. Upon reaching the site, the endoprosthesis is expanded, for example, so that it may contact the walls of the lumen.

The expansion mechanism may include forcing the endoprosthesis to expand radially. For example, the expansion mechanism may include the catheter carrying a balloon, which carries a balloon-expandable endoprosthesis. The balloon may be inflated to deform and to fix the expanded endoprosthesis at a predetermined position in contact with the lumen wall. The balloon may then be deflated, and the catheter withdrawn.

In another delivery technique, the endoprosthesis is formed of an elastic material that may be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

### SUMMARY

Medical devices such as endoprostheses are described herein. Generally stated, one aspect of the medical devices described herein features an endoprosthesis (for example, a stent), comprising a tubular member capable of maintaining patency of a bodily vessel. The tubular member includes a duplex stainless steel (e.g., 2205 stainless steel).

The tubular member may be substantially entirely made of the duplex stainless steel or less than the entire tubular member may be made of the duplex stainless steel. The tubular member may comprise a band and an elongated portion extending from the band (e.g., with the band having a lower yield strength than that of the elongated portion), and either the band, the elongated portion, or both, may be made of the duplex stainless steel.

In another embodiment, a medical device (for example in the form of an endoprosthesis, a wire, a cutting element configured to be supported by a medical balloon, or an orthopedic implant), includes a duplex stainless steel as described above in the general aspect and in the preferred embodiments.

A third aspect of this disclosure generally features a method of making a medical device, by forming a duplex stainless steel. The duplex stainless steel is then included in at least a portion of the medical device.

Embodiments may include one or more of the following advantages. Duplex stainless steels may include high contents of chromium and molybdenum, which may improve intergranular and pitting corrosion resistance, respectively. Duplex stainless steels may also include nitrogen. Not wishing to be bound by theory, nitrogen may promote structural hardening by interstitial solid solution mechanism. The two-phase microstructure of duplex stainless steel may provide higher resistance to pitting and stress corrosion cracking relative to conventional stainless steels. The duplex stainless steel may have enhanced hardness, yield strength, excellent corrosion resistance, and good biocompatibility. The duplex stainless steel may be used to make, for example, stents with thin walls. A thin-walled stent may be easily delivered through a tortuous path and may be implanted in a smaller bodily vessel. A thin wall also provides less blockage and allows more bodily fluid to flow unrestrictedly through the lumen of the stent. Wall thickness is an important factor for CT and fluoroscopic imaging. A thin-wall with relatively low radiopacity may provide an improved image that does not obscure other features. Lower radiopacity also may reduce blooming artifacts sometimes observed in images of high radiopacity implants, which obscures the area within and adjacent to the implant. The duplex stainless steels (e.g., 2205 stainless steel) may be used to make a variety of medical devices and medical components. For example, 2205 stainless steel (e.g., UNS S31803) exhibits high impact toughness and has high corrosion and erosion fatigue properties, lower thermal expansion, and higher thermal conductivity relative to austenitic stainless steel.

In one aspect, an endoprosthesis as described herein may include a tubular member capable of maintaining patency of a bodily vessel, the tubular member comprising a duplex stainless steel.

In some embodiments, the duplex stainless steel comprises iron, molybdenum, chromium, nickel and nitrogen, and optionally comprises one or more of carbon, phosphorus, silicon, manganese, and sulfur. In some embodiments, the duplex stainless steel comprises: 4.50 to 6.50 weight percent nickel; 0.08 to 0.20 weight percent nitrogen; 21.00 to 23.00 weight percent chromium; 2.50 to 3.50 weight percent molybdenum; not greater than 0.03 weight percent carbon; not greater than 0.030 weight percent phosphorus; not greater than 1.00 weight percent silicon; not greater than 2.00 weight percent manganese; not greater than 0.020 weight percent sulfur; and iron.

In some embodiments, the endoprosthesis comprises a stent.

In some embodiments, the tubular member comprises multiple layers, at least one of the layers comprising the duplex stainless steel.

In some embodiments, the duplex stainless steel is magnetized.

In some embodiments, the magnetized duplex stainless steel is magnetized and is capable of attracting and/or retaining cells comprising magnetic particles.

In another aspect, the medical devices described herein comprise a duplex stainless steel. In some embodiments, the medical device is in the form of an endoprosthesis. In some embodiments, the medical device is in a form selected from the group consisting of a wire, a cutting element configured to be supported by a medical balloon, an orthopedic implant, a vascular conduit, a load bearing joint, a sphincter, and a valve-based structure

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably.

The term “and/or” means one or all of the listed elements or a combination of any two or more of the listed elements.

The above summary is not intended to describe each embodiment or every implementation of the medical devices and methods described herein. Rather, a more complete understanding of the medical devices and methods described herein will become apparent and appreciated by reference to the following Description of Illustrative Embodiments and claims in view of the accompanying figures of the drawing.

### DESCRIPTION OF DRAWING

The FIGURE is a perspective view of one embodiment of an endoprosthesis (e.g., a stent).

### DESCRIPTION OF ILLUSTRATIVE EMBODIMENTS

In the following description of illustrative embodiments, reference is made to the accompanying drawing which forms a part hereof, and in which is shown, by way of illustration and the accompanying description, various embodiments of endoprostheses, e.g., stents and stent systems. It is to be understood that other embodiments may be utilized and structural changes may be made without departing from the scope of the present invention.

Referring to the FIGURE, an endoprosthesis (as shown, a stent 20) has the form of a tubular member 21 capable of maintaining the patency of a bodily lumen, such as a vascular vessel. In the depicted embodiment, tubular member 21 is defined by a plurality of bands 22 and a plurality of connectors 24 that extend between and connect adjacent bands. During use, bands 22 are expanded from an initial, small diameter to a larger diameter to contact stent 20 against a wall of a vessel, thereby maintaining the patency of the vessel. Connectors 24 provide stent 20 with flexibility and conformability that allow the stent to adapt to the contours of the vessel.

The tubular member 21 of stent 20 includes (e.g., is formed of) a duplex steel (e.g., type 2205 stainless steel, e.g., Carpenter 2205, etc.). Duplex steel (e.g., 2205 stainless steel) contains both ferritic and austenitic phases. Duplex steel may exhibit excellent corrosion resistance, high strength, and good biocompatibility. Duplex steel may be magnetizable and may, for example, magnetically attract and retain iron-labeled biological (e.g., endothelial) cells. For example, an austenitic/ferritic (21% chromium) steel may be magnetized by placement in, for example, a 2T MR magnet or a 3T MRI field, for a period of time (e.g., 30 minutes) to become magnetized.

Magnetized devices (e.g., stents, etc.) may attract labeled cells. For example, cells may be labeled with superparamagnetic particles (e.g., nanoparticles (i.e., having a dimension less than 1,000 nanometers)). Super paramagnetic particles may be biodegradable. Cells may be labeled with, for example, 200-nm PLGA-magnetite (poly(lactide-co-glycolide) magnetite) particles. In some embodiments, magnetic nanoparticles may be used to rapidly endothelialize magnetic devices (e.g., grafts), thereby facilitating healing and improving stent patency.

Examples of magnetic medical devices (e.g., implantable medical apparatuses), kits for magnetically coating and/or magnetizing a medical device, and methods for magnetically attaching and/or coating an implantable medical device, and methods for magnetizing and/or demagnetizing a medical device may be found in U.S. Patent Application Publication No. US 2009/0118817 (Sandhu et al.).

The use of a duplex stainless steel may, in some embodiments, allow stent 20 and other medical devices including the duplex stainless steel to be formed with thinner sections than could be the case for comparable devices made with other materials. For example, cardiovascular 3 mm diameter stent 20 including duplex stainless steel having the second composition may be formed with a thin wall (e.g., from about 0.0010 inch to about 0.0044 inch wall thickness). A thinner-walled stent may potentially be more easily delivered through a tortuous path and may potentially be implanted in a smaller bodily vessel, at least in part because a thinner-wall stent may have more axial flexibility than a thick-wall stent. A thinner wall may also provide less blockage and may allow more bodily fluid to flow unrestrictedly through the lumen of stent 20. Using less material for stent 20 also reduces the amount of foreign material placed in the body when the stent is implanted.

Duplex stainless steels have a mixed microstructure of austenite and ferrite, with the ratio (by weight) of austenite to ferrite ranging about 40/60 to about 60/40 (and in some embodiments preferably about 50/50). As discussed herein, duplex stainless steels may have improved strength over austenitic stainless steels and also improved resistance to localized corrosion, particularly pitting, crevice corrosion and stress corrosion cracking. They are typically characterized by high chromium (e.g., 19-28%) and molybdenum (e.g., up to 5%) and lower nickel contents than austenitic stainless steels. Duplex stainless steel grades are typically characterized into groups based on their alloy content and corrosion resistance. Lean duplex stainless steel refers to grades such as UNS S32101, S32304, and S32003. The standard duplex stainless steel is, in some embodiments, 22% chromium with S31803/S32205 (known as 2205) being the most widely used. Super duplex stainless steel typically refers to 25% chromium grades such as S32760 (Zeron 100), S32750 (2507), and S32550 (Ferralium). Hyper duplex stainless steel refers to higher chromium grades such as S32906. The properties of duplex stainless steels are achieved with an overall lower alloy content than similar-performing super-austenitic grades, making their use cost-effective for many applications.

The composition of a duplex stainless steel (e.g., 2205 stainless steel) typically includes chromium, molybdenum, nickel, nitrogen, iron, and one or more other elements, such as carbon, phosphorus, silicon, manganese, and sulfur.

In some embodiments, duplex stainless steel may or may not include carbon. In those embodiments that do include carbon, duplex stainless steel may include a carbon content of at most about 0.06 weight percent (in other embodiments, at most 0.040 weight percent and in still other embodiments, at most 0.030 weight percent).

In some embodiments, duplex stainless steel may include a manganese content of at most about 6.0 weight percent (in other embodiments, at most about 2.50 weight percent, in other embodiments, at most about 2.00 weight percent, in other embodiments, at most about 1.50 weight percent, in other embodiments, at most about 1.20 weight percent, and in still other embodiments, at most about 1.00 weight percent). In some embodiments, duplex stainless steel may or may not include a manganese content of at least about 0.0 weight percent. In some embodiments, duplex stainless steel may include a manganese content from about 4.0 weight percent to about 6.0 weight percent.

In some embodiments, duplex stainless steel may or may not include phosphorus. In those embodiments that do include phosphorus, duplex stainless steel may include a phosphorus content of at most about 0.045 weight percent (in other embodiments, at most about 0.040 weight percent, in other embodiments, at most about 0.035 weight percent, and in still other embodiments, at most about 0.030 weight percent).

In some embodiments, duplex stainless steel may or may not include sulfur. In those embodiments that do include sulfur, duplex stainless steel may include a sulfur content of at most about 0.030 weight percent (in other embodiments, at most about 0.020 weight percent and in still other embodiments, at most about 0.010 weight percent).

In some embodiments, duplex stainless steel may or may not include silicon. In those embodiments that do include silicon, duplex stainless steel may include a silicon content of at most about 1.0 weight percent (in other embodiments, at most about 0.80 weight percent and in still other embodiments, at most about 0.75 weight percent).

Chromium may enhance corrosion resistance, e.g., by increasing the pitting resistance of stainless steel. In some embodiments, duplex stainless steel may include a chromium content of at most about 28.0 weight percent (e.g., in other embodiments, at most about 27.0 weight percent, in other embodiments, at most about 26.0 weight percent, in other embodiments, at most about 24.5 weight percent, in other embodiments, at most about 23.0 weight percent, and in still other embodiments, at most about 21.5 weight percent). In some embodiments, duplex stainless steel may include a chromium content of at least about 19.5 weight percent (e.g., in other embodiments, at most about 21.0 weight percent, in other embodiments, at most about 21.5 weight percent, in other embodiments, at most about 22.0 weight percent, in other embodiments, at most about 23.0 weight percent, and in still other embodiments, at most about 24.0 weight percent). In some embodiments, duplex stainless steel may include a chromium content within the following ranges of weight percent: about 19.5 to about 21.5, about 21.0 to about 23.0, about 21.5 to about 24.5, about 22.0 to about 23.0, about 23.0 to about 28.0, about 24.0 to about 26.0, or about 24.0 to about 27.0.

In some embodiments, duplex stainless steel may include a nickel content of at most about 8.0 weight percent (in other embodiments, at most about 7.5 weight percent, in other embodiments, at most about 6.5 weight percent, in other embodiments, at most about 5.5 weight percent, in other embodiments, at most about 5.0 weight percent, and in still other embodiments, at most about 3.00 weight percent). In some embodiments, duplex stainless steel may include a nickel content of at least about 1.00 weight percent (in other embodiments, at least about 2.5 weight percent, in other embodiments, at least about 3.0 weight percent, in other embodiments, at least about 4.5 weight percent, in other embodiments, at least about 5.5 weight percent, and in still other embodiments, at least about 6.0 weight percent). In some embodiments, duplex stainless steel may include a nickel content within the following ranges of weight percent: about 1.00 to about 3.00, about 2.5 to about 5.0, about 3.0 to about 5.5, about 4.5 to about 6.5, about 5.5 to about 6.5, about 5.5 to about 7.5, about 5.5 to about 8.0, or about 6.0 to about 8.0.

Molybdenum may also be included in a duplex stainless steel to enhance the resistance of the material to corrosion, e.g., pitting and crevice corrosion. In some embodiments, duplex stainless steel may include a molybdenum content of at most about 5.0 weight percent (e.g., in other embodiments, at most about 5.0 weight percent, in other embodiments, at most about 4.0 weight percent, in other embodiments, at most about 3.9 weight percent, in other embodiments, at most about 3.5 weight percent, in other embodiments, at most about 2.0 weight percent, and in still other embodiments, at most about 0.60 weight percent). In some embodiments, duplex stainless steel may include a molybdenum content of at least about 0.0 weight percent (in other embodiments, at least about 0.05 weight percent, in other embodiments, at least about 1.0 weight percent, in other embodiments, at least about 1.20 weight percent, in other embodiments, at least about 2.5 weight percent, in other embodiments, at least about 2.9 weight percent, and in still other embodiments, at least about 3.0 weight percent). In some embodiments, duplex stainless steel may include a molybdenum content within the following ranges of weight percent: about 0.05 to about 0.60, about 1.0 to about 2.0, about 1.20 to about 2.00, about 2.5 to about 3.5, about 2.9 to about 3.9, about 3.0 to about 3.5, about 3.0 to about 4.0, or about 3.0 to about 5.0.

In some embodiments, duplex stainless steel may or may not include nitrogen. If included, nitrogen may be included in an amount of greater than 0.0 weight percent (in other embodiments, at least about 0.05 weight percent, in other embodiments, at least about 0.08 weight percent, in other embodiments, at least about 0.10 weight percent, in other embodiments, at least about 0.14 weight percent, in other embodiments, at least about 0.20 weight percent, and in still other embodiments, at least about 0.24 weight percent). At an upper end, some embodiments of duplex stainless steel may include a nitrogen content of at most about 0.035 weight percent (in other embodiments, at most about 0.32 weight percent, in other embodiments, at most about 0.30 weight percent, in other embodiments, at most about 0.25 weight percent, in other embodiments, at most about 0.20 weight percent, and in still other embodiments, at most about 0.17 weight percent). In some embodiments, duplex stainless steel may include a nitrogen content within the following ranges of weight percent: about 0.05 to about 0.17, about 0.05 to about 0.20, about 0.08 to about 0.2, about 0.10 to about 0.20, about 0.10 to about 0.25, about 0.14 to about 0.20, about 0.20 to about 0.30, about 0.20 to about 0.35, or about 0.24 to about 0.32.

In some embodiments, duplex stainless steel may or may not include copper. If included, copper may be included in an amount of at least about 0.0 weight percent (in other embodiments, at least about 0.05 weight percent, in other embodiments, at least about 0.20 weight percent, in other embodiments, at least about 0.50 weight percent, and in still other embodiments, at least about 1.5 weight percent). In some embodiments, duplex stainless steel may include a copper content of at most about 2.5 weight percent (in other embodiments, at most about 2.00 weight percent, in other embodiments, at most about 1.00 weight percent, in other embodiments, at most about 0.80 weight percent, in other embodiments, at most about 0.60 weight percent, and in still other embodiments, at most about 0.50 weight percent). In some embodiments, duplex stainless steel may include a copper content within the following ranges of weight percent: about 0.05 to about 0.60, about 0.20 to about 0.80, about 0.50 to about 1.00, about 0.50 to about 2.00, or about 1.5 to about 2.5.

The duplex stainless steels may include one or more microalloyed elements or residual amounts of impurities elements. For example, the duplex stainless steels may or may not include phosphorus (e.g., 0.030 wt % maximum), silicon (e.g. 1.00 wt % maximum), sulfur (e.g., 0.020 wt % maximum), manganese (e.g., about 2.00 wt %), and/or carbon (e.g., about 0.030 wt % maximum). Other microalloyed and residual elements are possible, which may be a function of the source of the materials.

Iron makes up the balance of the duplex stainless steels, e.g., after accounting for the other elements in the stainless steels described above. In certain embodiments, the stainless steels may include greater than 0 and less than about 72 weight percent of iron. For example, the stainless steels may include a maximum of less than or equal to about 72, 70, 68, or 66 weight percent of iron; and/or greater than or equal to about 64, 66, 68, or 70 weight percent of iron.

The duplex stainless steels may be synthesized by intimately combining the components of the stainless steels. The duplex stainless steels may also be formed by melting elemental powders in the appropriate concentrations. Melting may be performed using vacuum induction melting (VIM), vacuum arc remelting (VAR), electron beam melting (EBM), plasma melting, vacuum or inert gas plasma deposition. Solid state alloying may be performed using powder metals and hot isostatic pressing and/or cold pressing and sintering. Samples may be in the form of an ingot, a compact, or a deposit.

In some embodiments, duplex stainless steel includes low amounts of phases that may be detrimental to the properties of the duplex stainless steel. For example, 2205 stainless steel may include not greater than about 3,000 ppm (e.g., not greater than about 2,500 ppm, 2,000 ppm, 1,500 ppm, 1,000 ppm, 500 ppm, or 100 ppm) of carbon and not greater than 2,000 ppm (e.g., not greater than about 1,500 ppm, or 1,000 ppm) of nitrogen.

Duplex stainless steel may be characterized using microscopic methods and/or mechanical methods. As indicated above, duplex stainless steel includes distinct ferritic and austenitic phases, and as a result, the phases may be detected (e.g., mapped) using metallography techniques, such as scanning electron microscopy (SEM), auger electron microscopy (AES) and transmission electron microscopy (TEM). The compositions of phases may be determined by techniques such as energy dispersive spectroscopy (EDS) X-ray analysis.

Stent 20 may be formed by making a duplex stainless steel composition, forming the duplex stainless steel into a tubing, forming the tubing into a pre-stent, and finishing the pre-stent into stent 20

Next, bands 22 and connectors 24 of stent 20 are formed, for example, by cutting the tubing, to form a pre-stent. Selected portions of the tubing may be removed to form bands 22 and connectors 24 by laser cutting, as described in U.S. Pat. No. 5,780,807 (Saunders). In certain embodiments, during laser cutting, a liquid carrier, such as a solvent or an oil, is flowed through the lumen of the tubing. The carrier may prevent dross formed on one portion of the tube from re- depositing on another portion, and/or reduce formation of recast material on the tubing. Other methods of removing portions of the tubing may be used, such as mechanical machining (e.g., micro-machining), electrical discharge machining (EDM), and photoetching (e.g., acid photoetching).

In some embodiments, after bands 22 and connectors 24 are formed, areas of the pre-stent affected by the cutting operation above may be removed. For example, laser machining of bands 22 and connectors 24 may leave a surface layer of melted and resolidified material and/or oxidized metal that may adversely affect the mechanical properties and performance of stent 20. The affected areas may be removed mechanically (such as by grit blasting or honing) and/or chemically (such as by etching or electropolishing).

The pre-stent may then be finished. The pre-stent may be finished, for example, by electropolishing to a smooth finish. Because the pre-stent may be formed to near-net size, relatively little of the unfinished stent need to be removed to finish the pre-stent. As a result, further processing (which may damage the pre-stent) and costly materials may be reduced. In some embodiments, about 0.0001 inch of the pre-stent material may be removed by chemical milling and/or electropolishing to yield a stent.

Stent 20 may be of a desired shape and size (e.g., coronary stents, aortic stents, peripheral vascular stents, gastrointestinal stents, urology stents, and neurology stents). Depending on the application, stent 20 may have a diameter of between, for example, 1 mm to 46 mm. In certain embodiments, a coronary stent may have an expanded diameter of from about 2 mm to about 6 mm. In some embodiments, a peripheral stent may have an expanded diameter of from about 5 mm to about 24 mm. In certain embodiments, a gastrointestinal and/or urology stent may have an expanded diameter of from about 6 mm to about 30 mm. In some embodiments, a neurology stent may have an expanded diameter of from about 1 mm to about 12 mm. An abdominal aortic aneurysm (AAA) stent and a thoracic aortic aneurysm (TAA) stent may have a diameter from about 20 mm to about 46 mm. Stent 20 may be balloon-expandable, self-expandable, or a combination of both (e.g., U.S. Pat. No. 5,366,504 (Andersen et al.)).

In use, stent 20 may be used, e.g., delivered and expanded, using a catheter delivery system, such as a balloon catheter system. Catheter systems are described in, for example, U.S. Pat. Nos. 5,195,969 (Wang et al.), 5,270,086 (Hamlin), and 6,726,712 (Raeder-Devens). Stents and stent delivery are also exemplified by, e.g., the RADIUS or SYMBIOT systems, available from Boston Scientific Scimed, Maple Grove, Minn.

While a number of embodiments have been described above, the invention is not so limited. Stent 20 may be a part of a covered stent or a stent-graft. In other embodiments, stent 20 may include and/or be attached to a biocompatible, non-porous or semi-porous polymer matrix made of polytetrafluoroethylene (PTFE), expanded PTFE, polyethylene, urethane, or polypropylene.

Stent 20 may, in some embodiments, include a releasable therapeutic agent, drug, or a pharmaceutically active compound, such as described in U.S. Pat. Nos. 5,674,242 (Phan et al.), 6,676,987 (Zhong et al.), and 7,462,366 (Lanphere et al.). The therapeutic agents, drugs, or pharmaceutically active compounds may include, for example, anti-thrombogenic agents, antioxidants, anti-inflammatory agents, anesthetic agents, anti-coagulants, and antibiotics. In some embodiments, a stent may be formed by fabricating a wire including a duplex stainless steel, and knitting and/or weaving the wire into a tubular member.

While stent 20 is shown above as being formed wholly of duplex stainless steel, in other embodiments, the duplex stainless steel forms one or more selected portions of the medical device. For example, stent 20 may include multiple layers in which one or more layers include duplex stainless steel, and one or more layers do not include duplex stainless steel. Any such layers may, in some embodiments, be in the form one or more annular layers that extend around the circumference and along the length of the stent 20. Any layers of duplex stainless steel may be located on an interior surface of the stent (i.e., facing the lumen defined by the stent 20), on the exterior stent (i.e., facing away from the lumen defined by the stent 20), and/or in between the interior and exterior surfaces of the stent 20. Any layers that do not include duplex stainless steel may include one or more of the biocompatible materials, such as stainless steel, titanium, tantalum, platinum, iridium, or a shape memory material (e.g., nickel-titanium alloy). The layering of duplex stainless steel provides yet another way to tailor and tune the properties of the medical device. Stents including multiple layers are described, for example, in U.S. Pat. Appl. Pub. No. US 2004/0044397 (Stinson), and U.S. Pat. No. 6,287,331 (Heath).

In some embodiments, bands 22 and connectors 24 may include the same or different materials and may have the same or different material properties. For example, connectors 24 may have a higher hardness, and as a result, the connectors have a higher yield strength than the yield strength of bands 22. The high yield strength of connectors 24 allows them to have small cross-sectional sizes, which allow them to easily deform so that stent 20 may conform well to a vessel that is not straight. The yield strength and the section size are balanced to allow connectors 24 to easily deform while remaining resistant to fracture. In comparison, the low yield strength of bands 22 reduces elastic recoil when stent 20 is crimped to a delivery system and during in vivo expansion. The yield strength and the section size of bands 22 are balanced to provide good resistance to radial compression and to control elastic recoil.

Without wishing to be bound by theory, it is believed that stent 20 may experience relatively high levels of stress during use. For example, stent 20 may be bent as it tracks through a tortuous vessel during delivery, as it is expanded, and/or when it is placed in a curved vessel. After implantation, stent 20 may also experience stress from movement caused by a beating heart or by the subject's breathing. The stress that exceeds the yield strength of the material may strain the relatively narrow connectors 24 and fracture the connectors. A fractured connector may provide surfaces that disrupt blood flow and/or provide sites on which blood may aggregate and undesirably lead to blood clotting or thrombosis in the vessel. By enhancing the mechanical properties of connectors 24 (i.e., increasing the yield strength), the connectors may elastically tolerate the stress without leading to excessive straining that may lead to fracture, while still being easily deformable. At the same time, bands 22 are able to have good radial strength to support the vessel.

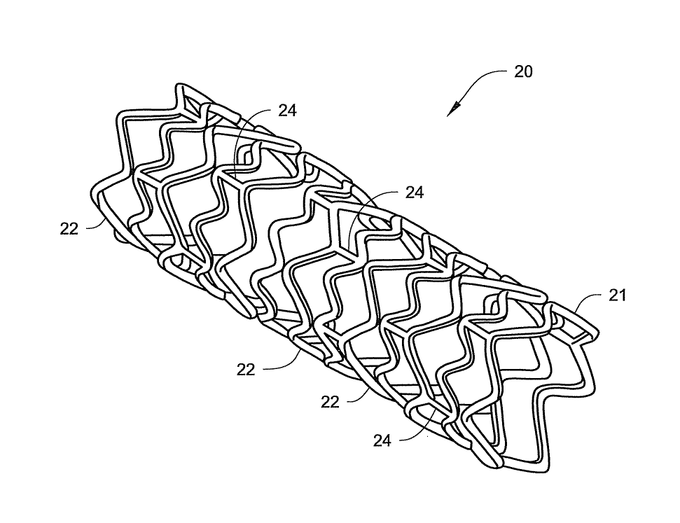

As used herein, a band 22 refers to a portion of a stent that extends circumferentially about the stent. The band may extend completely about the circumference of a stent, for example, such that the ends of the band are joined, or the band may extend partially about the circumference. The band may extend substantially linearly or nonlinearly, for example, in an undulating pattern or a zigzag pattern as shown in FIG. 1. In some embodiments, bands 22 are connected together by integrally formed connectors that extend between and transversely to the bands. As used herein, a connector 24 refers to a portion of a stent that extends from a band of the stent, for example, from a first band to an adjacent second band along the length of the stent. The connector may include one strut or a plurality of struts. The connector may extend linearly (e.g., parallel to the longitudinal axis of the stent) or nonlinearly, for example, in an undulating patter or zigzag pattern. Connectors 24 include one or more curved portions, examples of which are described in U.S. Pat. Nos. 6,656,220 (Gomez et al.); 6,629,994 (Gomez et al.); and 6,616,689 (Ainsworth et al.).

The duplex stainless steels described herein may be used to form other medical devices or components of medical devices. For example, the duplex stainless steels may be used to form wires (e.g., used to reinforce a guide catheter), cutting elements configured to be supported by a medical balloon, metal staples and wires used for wound closure, and an orthopedic implant, such as hip stems, screws, pins, plates and knee trays. Duplex stainless steels may also be used in a variety of implantable devices including, but not limited to, vascular conduits, load bearing joints, sphincters, and valve-based structures.

The complete disclosure of the patents, patent documents, and publications cited in herein are incorporated by reference in their entirety as if each were individually incorporated.

Illustrative embodiments of medical devices and methods are discussed and reference has been made to possible variations. These and other variations and modifications will be apparent to those skilled in the art without departing from the scope of the invention, and it should be understood that this invention is not limited to the illustrative embodiments set forth herein. Accordingly, the invention is to be limited only by the claims provided below and equivalents thereof.

1. An endoprosthesis comprising a tubular member capable of maintaining patency of a bodily vessel, the tubular member comprising a duplex stainless steel.

2. The endoprosthesis of claim 1, wherein the duplex stainless steel comprises iron, molybdenum, chromium, nickel and nitrogen.

3. The endoprosthesis of claim 2, wherein the duplex stainless steel comprises one or more of carbon, phosphorus, silicon, manganese, and sulfur.

4. The endoprosthesis of claim 1, wherein the duplex stainless steel comprises:
4.50 to 6.50 weight percent nickel;
0.08 to 0.20 weight percent nitrogen;
21.00 to 23.00 weight percent chromium;
2.50 to 3.50 weight percent molybdenum;
not greater than 0.03 weight percent carbon;
not greater than 0.030 weight percent phosphorus;
not greater than 1.00 weight percent silicon;
not greater than 2.00 weight percent manganese;
not greater than 0.020 weight percent sulfur; and
iron.

5. The endoprosthesis of claim 1, wherein the endoprosthesis comprises a stent.

6. The endoprosthesis of claim 1, wherein the tubular member comprises multiple layers, at least one of the layers comprising the duplex stainless steel.

7. The endoprosthesis of claim 1, wherein the duplex stainless steel is magnetized.

8. The endoprosthesis of claim 7, wherein the magnetized duplex stainless steel is capable of attracting and/or retaining cells comprising magnetic particles.

9. A medical device comprising duplex stainless steel.

10. The medical device of claim 9 in the form of an endoprosthesis.

11. The medical device of claim 9 in a form selected from the group consisting of a wire, a cutting element configured to be supported by a medical balloon, an orthopedic implant, a vascular conduit, a load bearing joint, a sphincter, and a valve-based structure.

12. The endoprosthesis of claim 1, wherein the duplex stainless steel comprises iron, molybdenum, chromium, nickel and nitrogen, and further wherein the duplex stainless steel comprises:
4.50 to 6.50 weight percent nickel;
0.08 to 0.20 weight percent nitrogen;
21.00 to 23.00 weight percent chromium;
2.50 to 3.50 weight percent molybdenum;
not greater than 0.03 weight percent carbon;
not greater than 0.030 weight percent phosphorus;
not greater than 1.00 weight percent silicon;
not greater than 2.00 weight percent manganese;
not greater than 0.020 weight percent sulfur; and
iron.

13. The endoprosthesis of claim 12, wherein the tubular member comprises multiple layers, at least one of the layers comprising the duplex stainless steel.

14. The endoprosthesis of claim 12, wherein the duplex stainless steel is magnetized.

15. The endoprosthesis of claim 14, wherein the magnetized duplex stainless steel is capable of attracting and/or retaining cells comprising magnetic particles.

16. The endoprosthesis of claim 1, wherein the duplex stainless steel comprises one or more of carbon, phosphorus, silicon, manganese, and sulfur, and further wherein the duplex stainless steel comprises:
4.50 to 6.50 weight percent nickel;
0.08 to 0.20 weight percent nitrogen;
21.00 to 23.00 weight percent chromium;
2.50 to 3.50 weight percent molybdenum;
not greater than 0.03 weight percent carbon;
not greater than 0.030 weight percent phosphorus;
not greater than 1.00 weight percent silicon;
not greater than 2.00 weight percent manganese;
not greater than 0.020 weight percent sulfur; and
iron.

17. The endoprosthesis of claim 16, wherein the tubular member comprises multiple layers, at least one of the layers comprising the duplex stainless steel.

18. The endoprosthesis of claim 16, wherein the duplex stainless steel is magnetized.

19. The endoprosthesis of claim 18, wherein the magnetized duplex stainless steel is
